(19) United States
(12) Patent Application Publication
(10) Pub. No.: US 2007/0148266 A1
(43) Pub. Date: Jun. 28, 2007

(54) Pine needle extract

(75) Inventors: Youchun Yan, Wormerveer (NL); Frederick William Cain, Wormerveer (NL); Ulrike Schmid, Wormerveer (NL); Wiro Stam, Wormerveer (NL); Jeanet Gerritsen, Wormerveer (NL)

Correspondence Address: MORGAN LEWIS & BOCKIUS LLP, 1111 PENNSYLVANIA AVENUE NW, WASHINGTON, DC, 20004, US

(73) Assignee: LODERS CROKLAAN B.V., Womerveer (NL)

(21) Appl. No.: 10/577,376

(22) PCT Filed: Oct. 29, 2004
(86) PCT No.: PCT/GB04/04580

§ 371 Date: Dec. 28, 2006

(30) Foreign Application Priority Data
Oct. 30, 2003 (EP) 03256868.5

(51) Int. Cl. A61K 36/13

(52) U.S. Cl. 424/770

(57) ABSTRACT

A composition which is obtainable as an extract from pine needles, having therapeutic activity and comprising isocupressic acid compounds in an amount of less than 0.01 wt % and further comprising one or more organic acids, can be used in foodstuffs, pharmaceutical compositions and food supplements.

This invention relates to a composition, to foodstuffs, food supplements or pharmaceutical compositions comprising the composition, to uses of the composition and to a process for producing the composition.

Pine needles are the leaves of plants of the Pinaceae family, including the genus Pinus. Certain types of pine needles are available in abundant supply and have been used for various purposes. Pine needle extracts have been described as being useful in specific beverages in JP 08107778 A and JP 07059538. Rice cakes containing pine needle extracts are described in JP 01218562 A.

A process for the extraction of taxol from pine needles is described in WO 94/15483.

High blood pressure (or hypertension) is known to be associated with many medical problems. High blood pressure directly increases the risk of coronary heart disease and stroke. High blood pressure most predominantly occurs in people over 35 years old but environmental and genetic factors and some medical conditions, such as diabetes mellitus, gout or kidney disease can lead to an increased risk of high blood pressure in people of all ages.

WO 98/28990 discloses a method of preparing food seasonings, food ingredients and food items using plant sterols and/or stanols together with raised levels of one or more of magnesium, calcium and potassium. Ingestion of the food is said to lead to a decrease in both cholesterol level and blood pressure.

There remains a need for active materials that can reduce high blood pressure, particularly naturally occurring materials that can be used as food supplements or in foodstuffs.

U.S. Pat. No. 6,329,000 discloses the use of certain pine needle extracts for treating various diseases including myocarditis, angina, arrhythmia, diabetes, senile dementia, sudden deafness and hypertension. The pine needle extracts are obtained by a relatively simple extraction process using water and alcohol as solvents.

U.S. Pat. No. 5,607,971 discloses the extraction of vasoactive lipids from Pinus ponderosa using methanol, diethyl ether and methylene chloride. The compounds isolated are esterified alkanediols.

U.S. Pat. No. 5,690,984 describes a beverage made from pine needles by boiling the needles in water at high pressure in a pressure vessel together with a mixture of other natural products.

Pine needles and their extracts may contain isocupressic acids. Isocupressic acids have been described as causing toxicity problems in beef cattle. It has been found that in U.S. Pat. No. 6,329,000, U.S. Pat. No. 5,607,971 and U.S. Pat. No. 5,690,984, a substantial amount of isocupressic acid remains in the extracts.

U.S. Pat. No. 5,466,453 teaches a method for improving the taste of a pine extract. U.S. Pat. No. 6,254,858 relates to a hair treatment composition containing, amongst other components, pine juices. U.S. Ser. No. 187,802 describes a disinfecting composition containing maple sugar and extracts of pine needles and juniper berries. The inventors believe that the products will contain substantial amounts of isocupressic acid.

It has surprisingly been found that isocupressic acids can be removed from pine needle extracts to form an extract which still exhibits therapeutic activity (such as the ability to lower blood pressure).

According to the present invention, there is provided a composition which is obtainable as an extract from pine needles, having therapeutic activity and comprising isocupressic acid compounds in an amount of less than 0.01 wt % (by weight based on the total weight of the composition) and preferably further comprising one or more organic acids (e.g., shikimic and/or quinic acid).

In another aspect, the invention provides a foodstuff (for example a dairy based food product), food supplement or pharmaceutical composition comprising a composition of the invention.

A further aspect of the invention is a method of improving one or more properties of a food product selected from hardness, texture, aeration, spreadability, oral properties, mouthfeel, flavour, colour, viscosity, ease of processing and health properties, which comprises incorporating into the food product a composition comprising one or more organic compounds, said composition being obtainable as an extract from pine needles. The properties are improved compared to an otherwise identical food product that does not contain the material.

The invention also provides a composition, foodstuff, food supplement, pharmaceutical composition or dairy based food product of the invention for use to lower blood pressure in mammals, particularly in humans.

In yet another aspect, the invention provides the use of a material comprising one or more organic compounds, said material being obtainable as an extract from pine needles, for improving one or more properties of a food product selected from hardness, texture, aeration, spreadability, oral properties, mouthfeel, flavour, colour, viscosity, ease of processing and health properties. The properties are improved compared to an otherwise identical food product that does not contain the material.

A yet further aspect of the invention is a process for producing a composition of the invention, which comprises the following steps:

treating pine needles with a solvent selected from water, organic solvents and mixtures thereof (preferably water), preferably at an elevated temperature of from 40° C. to 110° C., to form a first extract;

removing isocupressic acid compounds from the first extract, preferably by treatment with an ion exchange resin and/or activated carbon (preferably whilst the first extract is in aqueous solution, more preferably at an elevated temperature); and

optionally, filtering and concentrating the treated extract to obtain the composition as a powder or a concentrate. Preferably, prior to step (a), the pine needles are pretreated with a non-polar solvent (e.g, an alkane having from 4 to 10 carbon atoms, such as hexane), more preferably at a temperature of from 40° C. to 90° C. This pretreatment typically removes at least a part of the isocupressic acids.

The composition of the invention, and products comprising the composition is capable of lowering blood pressure in mammals, particularly in humans. Therefore, the invention also involves a method of lowering blood pressure (and/or treating hypertension) in a mammal which comprises the administration of a composition, foodstuff, food supplement, pharmaceutical composition or dairy based food product of the invention. The invention also involves the use of a composition, foodstuff, food supplement, pharmaceutical composition or dairy based food product of the invention in the manufacture of a composition for treating and/or preventing hypertension.

Compositions of the invention can be in the form of solids or liquids, including solutions, suspensions and dispersions. Preferably, the composition is in the form of a powder or an aqueous solution.

It is surprising that the composition of the invention retains therapeutic activity, even though it has been treated to the extent that it comprises isocupressic acid compounds in an amount of less than 0.01 wt %, because it has been found that isocupressic acid compounds have activity in lowering blood pressure. The term therapeutic activity in this context means usefulness in the treatment, inhibition or prevention of diseases or disorders. Diseases and disorders include, but are not limited to, high blood pressure (hypertension).

The invention may involve one or more of the following effects: lowering blood pressure; lowering systolic and/or diastolic blood pressure; reducing heart rate; reducing sympathetic nerve activity; reducing the chance of coronary heart disease; reducing the chance of aneurisms; reducing the chance of strokes; improving blood circulation; improving the cardiovascular system; improving blood vessel health; reducing stress on smooth muscle tissue; reducing the chance of chest pains; providing part of a healthy lifestyle; improving the chance of healthy circulation; reducing the effect of aging on the blood vessels; reducing cardiac stress; and improving recovery time after exercising.

The composition of the invention preferably contains isocupressic acid compounds in an amount of less than 0.005 wt %, more preferably less than 0.003 wt %, even more preferably less than 0.002 wt % such as less than 0.001 wt %. The terms “isocupressic acid compounds” and “isocupressic acids” are used synonymously herein and refer to isocupressic acid itself and preferably related diterpene acids found in pine needles and their extracts, such as imbricatoloic acid, agathic acid, dihydroagathic acid and tetrahydroagathic acid. Isocuppressic acids may be in the form of derivatives of these acids, for example, acetylimbricatoloic acid and acetylisocupressic acid. Preferably, therefore, the composition of the invention contains isocupressic acid, imbricatoloic acid, agathic acid, dihydroagathic acid, tetrahydroagathic acid, acetylimbricatoloic acid and acetylisocupressic acid in an amount of less than 0.005 wt %, more preferably less than 0.003 wt %, even more preferably less than 0.002 wt % such as less than 0.001 wt %. Preferably, the composition is free of isocupressic acids or substantially free of isocupressic acids (i.e., to the extent that the presence of isocupressic acids cannot be detected by conventional techniques and/or has no effect on the properties of the composition). The level of isocupressic acids can be determined, for example, by GCMS.

The composition is obtainable, and is preferably obtained, from pine needles. Pine needles are preferably from species of pine other than Pinus ponderosa. Pine species include Pinus albicaulis, Pinus aristata, Pinus attenuata, Pinus balfouriana, Pinus banksiana, Pinus bungeana, Pinus cembra, Pinus cembroides, Pinus clausa, Pinus contorta, Pinus coulteri, Pinus densiflora, Pinus echinata, Pinus edulis, Pinus elliottii, Pinus engelmannii, Pinus flexilis, Pinus glabra, Pinus heldreichii, Pinus jeffreyi, Pinus lambertiana, Pinus longaeva, Pinus massoniana, Pinus monophylla, Pinus monticola, Pinus mugo, Pinus muricata, Pinus nigra, Pinus palustris, Pinus parviflora, Pinus pungens, Pinus quadrifolia, Pinus radiata, Pinus resinosa, Pinus rigida, Pinus sabiniana, Pinus serotina, Pinus strobiformis, Pinus strobus, Pinus sylvestris, Pinus tabulaeformis, Pinus taeda, Pinus thunbergiana, Pinus torreyana, Pinus virginiana, Pinus yuannensis and Pinus washoensis. Preferably, the composition is from Pinus massoniana, Pinus tabulaeformis or Pinus yuannensis, more preferably, the material is from Pinus massoniana. The composition preferably comprises one or more organic compounds, more preferably two or more organic compounds. Organic compounds are compounds that comprise carbon, hydrogen and oxygen atoms and optionally other atoms such as nitrogen, phosphorus and sulphur.

The composition preferably comprises at least 2 components A and B, wherein A is a compound that is obtainable from a mixture of A and B by elution from a silica column using 100% methanol as eluent and B is a compound obtainable from the same silica column using methanol/water mixtures (5-40% by volume) in a series of subsequent elutions. A is preferably selected from the group consisting of phytosterol, polyphenols, bioflavonoids, tannins, organic acids and their complexes. B is preferably selected from the group consisting of amino acids, peptides, proteins, quercetin, terpenoids, flavonol glycosides, biflavones, proanthocyanidins, polyprenols, lignans and minerals. The composition may comprise one or more compounds A and one or more compounds B.

Preferably, the composition comprises A (or total A compounds where more than one A compound is present) in an amount of from 5 to 60 wt %, preferably 10 to 50 wt %, most preferably 15 to 40 wt %, and the composition comprises B (or total B compounds where more than one B compound is present) in an amount of from 1 to 15 wt %, preferably 2 to 12 wt %, most preferably 3 to 10 wt %, based on the weight of the composition.

Therefore, in one embodiment, the composition of the invention comprises at least one compound selected from the group consisting of phytosterol, polyphenols, bioflavonoids, tannins, organic acids and their complexes and at least one compound selected from the group consisting of amino acids, peptides, proteins, quercetin, terpenoids, flavonol glycosides, biflavones, proanthocyanidins, polyprenols, lignans and minerals.

Compositions of the invention preferably comprise shikimic acid and/or quinic acid. Shikimic acid is preferably present in the compositions of the invention in an amount by weight of the composition of at least 10%, preferably at least 12%, more preferably at least 15%, such as at least 16%, at least 17%, at least 18%, at least 19% or at least 20%. The upper limit for the amount of shikimic acid is typically no more than 50%, more preferably no more than 40%, such as no more than 30%, or no more than 25%, again by weight of the composition. The shikimic acid may be present as a salt or other derivative, such as an acetyl ester.

Quinic acid (typically as D-quinic acid) is preferably present in the compositions of the invention in an amount by weight of the composition of at least 5%, preferably at least 6%, more preferably at least 7%, such as at least 8%, at least 9%, at least 10%, at least 12% or at least 15%. The upper limit for the amount of quinic acid is typically no more than 30%, more preferably no more than 27%, such as no more than 25%, or no more than 20%, again by weight of the composition. The quinic acid may be present as a salt or other derivative, such as an acetyl ester.

Specific preferred compositions of the invention comprise shikimic acid and quinic acid in the amounts specified in the preceding two paragraphs. An example of such a composition comprises at least 10% by weight shikimic acid and at least 5% by weight quinic acid, such as 10% to 30% shikimic acid and 5% to 20% quinic acid.

Compositions of the invention may further comprise low molecular weight sugars, preferably sugars having a molecular weight below 1000 Daltons. Preferred low molecular weight sugars are monosaccharide units and include glucose, fructose and inositol. The amount of low molecular weight sugars is preferably from 15% to 50% by weight, such as 20% to 40%, or 20% to 35% by weight of the composition.

Compositions of the invention may be used in a foodstuff (for example a dairy based food product), food supplement or pharmaceutical composition. These products provide a convenient form in which to deliver the composition. Compositions of the invention may comprise an antioxidant in an amount effective to increase the stability of the composition with respect to oxidation and optionally colouring agents and/or preservatives.

A preferred composition according to the invention is a foodstuff. Foodstuffs include liquids (e.g, beverages) and solids. Suitably, foodstuffs will be packaged and labelled as foodstuffs. Conventional foodstuffs may incorporate the composition of the invention in a suitable amount.

Pharmaceutical compositions may, for example, be in the form of tablets, pills, capsules, caplets, multiparticulates including: granules, beads, pellets and micro-encapsulated particles; powders, elixirs, syrups, suspensions and solutions. Pharmaceutical compositions will comprise a pharmaceutically acceptable diluent or carrier. Pharmaceutical compositions are preferably adapted for administration parenterally (e.g., orally). Orally administrable compositions may be in solid or liquid form and may take the form of tablets, powders, suspensions and syrups. Optionally, the compositions comprise one or more flavouring and/or colouring agents. Pharmaceutically acceptable carriers suitable for use in such compositions are well known in the art of pharmacy. The pharmaceutical compositions of the invention may contain 0.1-99% by weight of the composition of the invention. Pharmaceutical compositions of the invention are generally prepared in unit dosage form.

Further examples of product forms that comprise the composition are food supplements, such as in the form of a soft gel or a hard capsule, preferably comprising an encapsulating material selected from the group consisting of gelatin, starch, modified starch, starch derivatives such as glucose, sucrose, lactose and fructose. The encapsulating material may optionally contain cross-linking or polymerizing agents, stabilizers, antioxidants, light absorbing agents for protecting light-sensitive fills, preservatives and the like. Preferably, the unit dosage of the composition of the invention in the food supplements is from 1 mg to 1000 mg (more preferably from 100 mg to 750 mg).

The foodstuff of the invention is preferably a dairy based food product. The dairy based food product may contain the composition in an amount of from 0.01 wt % to 10 wt %, more preferably from 0.05 wt % to 5 wt %, even more preferably from 0.1 wt % to 2 wt %, based on the total weight of the dairy based food product and based on the dry weight of the composition. Dairy based food products include edible products comprising one or more proteins, fats and/or sugars derived from milk. Milk proteins include, for example, casein and milk sugars include, for example, lactose. Preferably, the dairy based food product comprises milk proteins in an amount of at least 0.01% by weight, more preferably 0.1% by weight, even more preferably 1% by weight, based on the weight of the dairy based food product.

Dairy based food products of the invention preferably have a water content of from 0.5 to 99.5 wt %, preferably 20 to 90 wt %, most preferably 30 to 85 wt %.

Dairy based food products are typically oil in water (O/W) emulsions, bicontinuous emulsions or duplex W/O/W (water in oil in water) emulsions.

Certain dairy based food products of the invention comprise a fat phase. The fat phase preferably displays a solid fat content (measured by NMR on a non-stabilised fat) at 5° C.(=N5) of >10, preferably >20, and at 35° C.(=N35) of <20, preferably <10, most preferably less than 5. Methods for determining solid fat content by NMR on non-stabilised fat are well known to those skilled in the art and include the method described in Fette, Seifen, Anstrichmittel, 80 (1978), 180-186. Non-stabilised means that the N-value is measured after first melting the fat above 80° C., whereupon the melt is cooled to 0° C. and kept at 0° C. for 30 minutes, then the fat is heated to the measurement temperature and kept at that temperature for 30 minutes, whereupon the N-value is measured.

Examples of dairy based food products of the invention are cream, milk, water continuous or bicontinuous spreads, confectionery or sweet spreads, chocolate, snack bars, nutritional bars, ice cream, confectionery fillings or toppings, bakery fillings or toppings, yoghurt, including drinkable yoghurt, curd cheese, milk shake, slimming drinks, cheese and cheese spreads.

Preferably, the dairy based food products of the invention are essentially free of trans fatty acids (which are carboxylic acids containing from 12 to 24 carbon atoms and having one carbon-carbon double bond) i.e, they contain trans fatty acids in an amount of less than 1% by weight, preferably less than 0.5% by weight, more preferably less than 0.1% by weight, such as less than 0.05% or less than 0.01% by weight.

The dairy based food product of the invention preferably has one or more of the following properties compared to a corresponding product that does not contain the composition: improved hardness, improved texture, improved aeration, improved spreadability, improved oral properties, improved mouthfeel, improved flavour, better colour, improved viscosity, better shape retention, improved whipping properties and improved ease of processing. The properties are improved compared to an otherwise identical food product that does not contain the material. Preferred properties that are improved according to the invention are oral properties and/or visual appearance, in particular increased similarity to butter in terms of oral properties and/or visual appearance.

The following non-limiting examples illustrate the invention and do not limit its scope in any way. In the examples and throughout this specification, all percentages, parts and ratios are by weight unless indicated otherwise.

### EXAMPLES

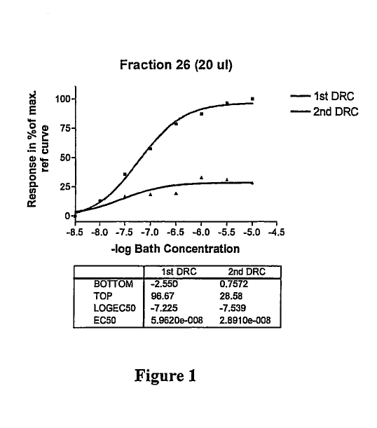

Reference is made in the examples to FIG. 1.

FIG. 1 shows the dose dependent contraction of rat aorta caused by phenylephrine and the inhibition of this effect by a pine needle extract of the invention.

### Example 1

Pine Needle Extraction

100 g pine needles from Pinus massoniana (isocupressic acids (ICA) content 0.33 wt %) were cleaned with water, cut into small pieces (3˜4 cm) and put in a flask. 500 g hexane was added to the flask and heated under stirring to reflux (˜60° C.) for about 3 to 5 h. The resulting pine needle solution was filtered through a Büchner funnel and the hexane removed using a rotary evaporator. The crude extract contains 8 wt % of compounds of the isocupressic acid family. This extract was not used for further experiments.

The residue which was left after treatment with hexane was transferred to a flask and 500 ml demineralised water was added. The mixture was stirred at 100° C. for about 3-5 h. Then the extract was filtered through a Büchner funnel and concentrated to 150 ml. To this extract 12.5 g resin (Dowex Marathon A, Polysep Industrial Consultants) was added; the temperature was maintained at 50° C. for 3 h. After filtration through a Büchner funnel to remove the resin, the solution was dried in a rotary evaporator to produce the pine needle powder (ICA content 0.003 wt %).

### Example 2

Thoracic aortas were obtained from spontaneous hypertensive rats (SHR). The thoracic aorta is cut into rings of 4 to 6 mm in length and each ring is connected to a tension transducer in a thermostatically controlled and oxygenated organ bath containing modified Krebs-Henselheit buffer. The contractions of rings of aorta are recorded continuously under isotonic conditions. After equilibrating the tissues, a single dose of 1 μM phenylephrine was given to sensitise the tissue, followed by washout. Hereafter, two cumulative dose response curves of phenylephrine were generated. The first dose response curve was obtained in the absence of an extract and served as a control curve. After thorough washing (7 times) the tissues were incubated with the pine needle extract for 1 hour. Following this incubation period, a second dose response curve was obtained in the presence of a concentrated form of the extract. The data were analysed taking the maximal response of the reference curve as a control.

FIG. 1 shows that phenylephrine causes a dose dependent contraction of rat aorta (the upper curve in the FIGURE). After incubation with pine needle extract of Example 1, the contraction of rat aorta by phenylephrine is clearly inhibited (the lower curve in the FIGURE).

### Example 3

Preparation of Extracts

Two pine needle extracts were produced. The first extract (Extract 1) is a comparative example containing isocupressic acids. The second example (Extract 2) is a purified form of the first extract and is an example according to the invention.

Extract 1 was obtained as follows:
100 g pine needles from Pinus massoniana was cleaned, cut into small pieces (about 3 to 4 cm) and put into a flask
1 litre water was added
The mixture was heated and maintained at reflux temperature (about 100° C.) for 5 h.
The pine needle residue was removed from the mixture
The pine needle extract (Extract 1) was obtained by removing water using a rotary evaporator.

Extract 2 was obtained by further treating Extract 1 as follows:
200 g pine needle extract obtained above as Extract 2 (containing 5-10% water) was mixed with 1.2 l demineralised water. This was allowed to stand in a water bath at 70° C. for half and hour to dissolve.
The mixture was transported to a reaction vessel and stirred for 15 minutes at 70° C.
40 g resin (Dowex 50 W) was added and stirring was continued for three hours at 50° C., the mixture was then filtered. After the filtration, this step (i.e., addition of resin, stirring and filtering) was repeated.
0.8 g of Norit SA4 active carbon was added.
The resulting mixture was stirred for 1 hour while keeping the temperature at 85° C.
The resulting mixture was filtered 3 times through a Büchner filter (54; Ø185 mm).
Water was evaporated from the filtrate using a rotary evaporator to obtain Extract 2.

The general procedure for the analytical method for determining isocupressic acid (ICA) is as follows:

The sample is extracted with 50 mL methylene chloride for about three hours by means of a method based on Soxhlet extraction. After extraction, the mehylene chloride is removed via rotary evaporation. After this step, heptadecanoic acid is added as an internal standard. This component is used to be able to quantify in the end. The samples are then dissolved in a little methylene chloride and put on a SPE column containing 500 mg of aminopropyl sorbent, which has been conditioned with the appropriate solvents beforehand. After the sample has been put on the SPE column the possible present “neutral” components are eluted with a solvent containing 9:1 by volume diether ether: methanol. After this clean-up step, the acids are eluted with the same solvent but containing an additional 1% of acetic acid. After removal of the solvent in a heating block, the sample is derivatized by means of 2 M diazomethane in ether (making methyl esters of the carboxylic acid groups) and by using MSTFA (N-methyl-N-trimethylsilyltrifluoroacetamide) for silylating any free hydroxyl groups left. This derivatisation step is done in sequence. After derivatisation the sample is properly diluted using isooctane and 1 μl is injected into the GC-MS. The GC-MS uses a 30 m CP-Sil 5 column (DB-1) with an internal diameter of 0.25 mm to separate the components. Temperature programming is used: 100 C, 1 minute hold ramped to 200 C at 40° C./min after which the temperature is ramped to 250° C. at 2° C./min. The helium carrier gas flow is kept constant at 1.0 mL/min(=+/−68 kPa gauge pressure at 100° C.). Splitless injection is used and the temperature of the injector is kept constant at 250° C. MS transfer line is also kept constant at 250° C. The MS detector was set to EI mode with an ionization energy of 70 eV. The mass range collected was from 50 to 550 Da using an electron multiplier voltage of 1250 V.

Blood Pressure Lowering Effects of Pine Needle Extracts:

Reference is made in this example to Table 1.

Table 1 shows the effect of pine needle extract on
the median effective concentration (EC₅₀) of phenylephrine and the difference between DRC1 and DRC2
maximal effect (Eₘₐₓ) relative to phenylephrine; the lower the value the more effective the extract

Isolated aorta rings from spontaneously hypertensive rats (SHR rats) were used as a model for testing vasoactive effects. Thoracic aortas were obtained from the SHR rats. The thoracic aorta was cut into rings of 4 to 6 mm in length and each ring was then connected to a tension transducer in a thermostatically controlled and carbogentaed organ bath containing modified Krebs-Henseleit buffer. The contractions of rings of aorta were recorded continuously under isotonic conditions. After equilibrating the tissues, a single dose of 1 μM phenylephrine was given to sensitise the tissue, followed by washout. Phenylephrine is an α-adenergic agonist, and is used in this experiment to induce contraction of the aorta rings. Hereafter, two cumulative dose response curves (DRC1 and DRC2) of phenylephrine were generated. DRC1 was obtained in the absence of an extract and served as a control curve, while DRC2 was obtained after incubation with 50 μg/mL pine needle extract. After thorough washing the tissues were incubated with the pine needle extract for 5 minutes. Following this incubation period, a second dose response curve was obtained in the presence of a concentrated form of the extract. The data were analysed talking
the median effective concentration (EC₅₀)
maximal effect (Eₘₐₓ) relative to phenylephrine

The following extracts were tested:
1. Pinus massoniana before Norit/Ion-exchange resin treatment (Extract 1)
2. Pinus massoniana after Norit/Ion-exchange resin treatment (Extract 2)
3. Comparative example: Extract according to U.S. Pat. No. 6,329,000 B1 (Ji Ling)
4. Comparative example: Extract according to U.S. Pat. No. 5,690,984 (Lim Jung Geun)
5. Comparative example: Ether extract according to U.S. Pat. No. 5,607,971 (Al-Mahmoud Mohsen)
6. Comparative example: Methylene chloride extract according to U.S. Pat. No. 5,607,971 (Al-Mahmoud Mohsen)

7. Comparative example: Methanol extract according to U.S. Pat. No. 5,607,971 (Al-Mahmoud Mohsen)

TABLE 1

| | | | | | | D- | | |
|---|---|---|---|---|---|---|---|---|
| | | | | | | quinic | | |
| | logEC₅₀ | logEC₅₀ | Difference | | ICA | aicid | Shikimic | LMW |
| Extracts | control | extract | logEC₅₀ | Eₘₐₓ | family¹ (%) | (%) | acid (%) | sugars² |
| 1 | −7.35 | −6.31 | −1.04 | 54 | 0.30 | 9.8 | 19.8 | 26.0 |
| 2 | −6.81 | −6.08 | −0.73 | 79 | 0.00 | 15.3 | 21.0 | 27.0 |
| 3 | −7.25 | −6.49 | −0.76 | 53 | 0.02 | 4.0 | 17.0 | 23.0 |
| 4 | −7.21 | −6.88 | −0.34 | 88 | | | | |
| 5 | −7.17 | −6.43 | −0.74 | 74 | 15.33 | 0.3 | 0.6 | 0.8 |
| 6 | −7.36 | −6.16 | −1.20 | 76 | 13.68 | 0.2 | 0.0 | 0.4 |
| 7 | −7.24 | −6.47 | −0.77 | 90 | 1.93 | 1.0 | 2.3 | 12.5 |

¹ICA family consists of dihydroagathic acid, imbricatoloic acid, isocupressic acid, agathic acid, acetylimbricatoloic acid and acetylisocupressic acid.
²Low molecular weight sugars are monosaccharide units and include glucose, fructose and inositol structures.

After incubation with Extract 1 the contraction of the rat aorta by phenylephrine is clearly inhibited (Emax 54). Extract 2 shows after removal of isocupressic acids by Norit treatment and ion exchange still activity (Emax 79) similar to extracts 5, 6, and 7 which are high in isocupressic acids.

### Example 4

Preparation of Ice Cream

Two ice cream products were prepared according to the following recipe and procedure.

Recipe

| | | With pine needle |
|---|---|---|
| | | extract of the |
| Ingredient | Reference (%) | invention (%) |
| Palm oil (POf iv 65) | 10 | 9.5 |
| Pine needle extract | 0 | 0.5 |
| Skimmed milk powder | 10 | 10 |
| Glucose syrup BS0424303 | 4 | 4 |
| Crystal sugar | 12 | 12 |
| Dextrose | 2 | 2 |
| Sorbitan monostearates | 1 | 1 |
| Water | 61 | 61 |

Procedure
Mix sugar, milk powder and dextrose
Add to water and mix
Heat mixture up to 70° C. in microwave
Add glucose syrup, palm oil, emulsifier and pine needle extract
Stir 4 minutes at room temperature with ultra-turrax at speed 9500
Place in bowl with ice-water and continue use of ultra-turax till temperature reach 21° C.
Leave emulsion overnight in refrigerator at 7° C.
Place ice machine in freezer overnight
Stir the emulsion for 40 minutes in ice machine
Observe during process and store samples in refrigerator −21° C.
   Evaluation

The ice cream with pine needle extract was found to maintain its shape over a longer period indicating that the pine needle extract has a positive effect on the microstructure of the emulsion.

### Example 5

Preparation of Muesli Bar

Recipe

Binding mixture

| | |
|---|---|
| Fat: Centremelt ES - HC 110 P 04* | 40% |
| yoghurt powder | 4% |
| Full cream milk powder 7% | |
| Skimmed milk powder | 5% |
| sugar | 30% |
| dextrose | 14% |
| lecithin | 0.4% |

*from Loders Croklaan BV

Coating

| | |
|---|---|
| Fat Couva 500 HD495P04* | 32% |
| Full cream milk powder | 10% |
| Skimmed milk powder | 10% |
| Sugar | 48% |
| Lecithin | 0.4% |
| Cream Vanillin DU-00569 | 0.03% |

*from Loders Croklaan BV

Preparation of the Muesli Bar

Prepare binding mixture:
crush 700 gram (35%) muesli into fine particles
add 200 grams (10%) rice crisps
mix with 1100 gram (55%) binding mixture

Based on approximately 20 gram binding for a coated bar at 28 gram, the following dosages were mixed:

| | Extract/binding | Concentration per bar |
|---|---|---|
| Reference | 0 | 0% |
| Pine needle extract | 14 g/300 g | 3% |
| | 14 g/200 g | 5% |

press the mixture in the moulds
put the moulds in the cooling cabinet until a solid bar is formed
de-mould
coat the bars with white couva 500 coating
cool in the cooling cabinet

Addition of the pine needle extract had no adverse effect on the process.

1. Composition which is obtainable as an extract from pine needles, having therapeutic activity, comprising isocupressic acid compounds in an amount of less than 0.01 wt % and further comprising one or more organic acids.

2. Composition according to claim 1, which contains isocupressic acid compounds in an amount of less than 0.005 wt %, preferably less than 0.003 wt %.

3. Composition according to claim 1 or claim 2 comprising at least two components A and B, wherein compound A is selected from the group consisting of phytosterol, polyphenols, bioflavonoids, tannins, organic acids and their complexes and compound B is selected from the group consisting of amino acids, peptides, proteins, quercetin, terpenoids, flavonol glycosides, biflavones, proanthocyanidins, polyprenols, lignans and minerals.

4. Composition as claimed in claim 1 or claim 2 which comprises a compound A selected from the group consisting of phytosterol, polyphenols, bioflavonoids, tannins, organic acids and their complexes and a compound B selected from the group consisting of amino acids, peptides, proteins, quercetin, terpenoids, flavonol glycosides, biflavones, proanthocyanidins, polyprenols, lignans and minerals.

5. Composition according to claim 4, wherein A is present in an amount of from 5 to 60 wt %, preferably 10 to 50 wt %, most preferably 15 to 40 wt %, and B is present in an amount of from 1 to 15 wt %, preferably 2 to 12 wt %, most preferably 3 to 10 wt %, based on the weight of the composition.

6. Composition according to claim 1 which comprise shikimic acid in an amount of from 10% to 50% by weight of the composition.

7. Composition according to claim 1 which comprises quinic acid in an amount of from 5% to 30% by weight of the composition.

8. Foodstuff, food supplement or pharmaceutical composition comprising a composition of claim 1.

9. Dairy based food product comprising a composition as claimed in claim 1.

10. Dairy based food product according to claim 9 having a water content of from 0.5 to 99.5 wt %, preferably 20 to 90 wt %, most preferably 30 to 85 wt %.

11. Dairy based food product according to claim 9 or claim 10 which is an oil in water (O/W) emulsion, a bicontinuous emulsion or a duplex W/O/W emulsion.

12. Dairy based food product according to claim 9, which is a cream, milk, water continuous or bicontinuous spread, confectionery or sweet spread, chocolate, snack bar, nutritional bar, ice cream, confectionery filling or topping, bakery filling or topping, yoghurt, drinkable yoghurt, curd cheese, milk shake, slimming drink, cheese or cheese spread.

13. Dairy based food product according to claim 9, comprising a fat phase that displays a solid fat content (measured by NMR on a non-stabilised fat) at 5° C.(=N5) of >10, preferably >20, and at 35° C.(=N35) of <20, preferably <10, most preferably less than 5.

14. Dairy based food product as claimed in claim 9 which is essentially free of trans fatty acids.

15. Dairy based food product according to which comprises from 0.05 wt % to 10 wt % of the composition of claim 1.

16. Dairy based food product according to claim 9 which has one or more of the following properties compared to a corresponding product that does not contain the composition improved hardness, improved texture, improved aeration, improved spreadability, improved oral properties, improved mouthfeel, improved flavour, better colour, improved viscosity, improved whipping properties and improved ease of processing.

17. (canceled)

18. (canceled)

19. A method of lowering blood pressure in a mammal, particularly a human, which comprises providing the mammal with an effective amount of a composition as claimed in claim 1, a foodstuff, food supplement or pharmaceutical composition as claimed in claim 8 or a dairy based food product as claimed in claim 9.

20. A method of improving one or more properties of a food product selected from hardness, texture, aeration, spreadability, oral properties, mouthfeel, flavour, colour, viscosity, shape retention, ease of processing and health properties, which comprises incorporating into the food product a composition comprising one or more organic compounds, said composition being obtainable as an extract from pine needles, having therapeutic activity and comprising isocupressic acid compounds in an amount of less than 0.01 wt % and further comprising one or more organic acids.

21. (canceled)

22. Process for producing the composition of claim 1, which comprises the following steps:
a. Treating pine needles with a solvent selected from water, organic solvents and mixtures thereof, to form a first extract;
b. Removing isocupressic acid compounds from the first extract, preferably by treatment with an ion exchange resin; and
c. Optionally, filtering and concentrating the treated extract to obtain the composition as a powder or a concentrate.

23. Process as claimed in claim 22, wherein prior to step (a), the pine needles are pretreated with a non-polar solvent.
